(19) United States
(12) Patent Application Publication
(10) Pub. No.: US 2025/0235643 A1
(43) Pub. Date: Jul. 24, 2025

(54) SYSTEM FOR MEASURING APPROPRIATE VENTILATORY PARAMETERS DURING TRAINING

(71) Applicant: Lumis Corp., Pittsburgh, PA (US)

(72) Inventors: Nehal J. Bhojak, Pittsburgh, PA (US); Douglas A. Nelson, JR., Pittsburgh, PA (US); Ryan M. Torbic, Pittsburgh, PA (US)

(73) Assignee: Lumis Corp., Pittsburgh, PA (US)

(21) Appl. No.: 19/028,796

(22) Filed: Jan. 17, 2025

Related U.S. Application Data
(60) Provisional application No. 63/622,332, filed on Jan. 18, 2024.

(51) Int. Cl. A61M 16/00

(52) U.S. Cl. CPC A61M 16/024; A61M 2016/0027; A61M 2016/0036; A61M 2205/3368; A61M 2205/502; A61M 2205/581; A61M 2205/584

(57) ABSTRACT

A system for measuring appropriate ventilatory parameters during training that includes one or more sensors, a memory unit that stores the sensor data, a processing unit that computes the data collected to provide real-time feedback on performance. The sensors may measure temperature, volume of airflow, ventilatory rate, ventilatory pressure, and other relevant properties. The stored data includes historical measurements collected over a period of time by the sensors. The processing unit computes the stored sensor data into performance metrics, compares the metrics against objective measures, and determines what real-time feedback to provide to the user so that the user can improve their skills. This feedback may be provided to the user through a multitude of auditory and visual methods. The system may include a range of standard input and output fittings so that it can be used with any common ventilatory or other airway devices during practice.

### RELATED APPLICATION

This application claims priority under 35 U.S.C. § 119 (e) to U.S. provisional patent application, U.S. Ser. No. 63/622,332, filed Jan. 18, 2024, which is herein incorporated by reference in its entirety.

### BACKGROUND

The subject matter discussed in the background section should not be considered prior art merely because of its mention in the background section. Similarly, a problem mentioned in the background section or associated with the subject matter of the background section should not be considered to have been previously recognized in the prior art. The subject matter in the background section merely represents different approaches, which in and of themselves, may also correspond to claimed embodiments.

During patient resuscitation, healthcare providers must be able to deliver adequate ventilation to the patient in order to administer oxygen and remove carbon dioxide in a safe manner. There are a number of medical devices and clinical techniques used to provide ventilation during resuscitation, and providers must be proficient with these devices and techniques. Regardless of device or technique, providers must know how to administer the appropriate volume, rate, and pressure of ventilations to the patient during resuscitation to give the patient the best chance of survival.

Providers develop proficiency during training with patient simulators while using the same medical devices and clinical techniques that they may use while providing care to real patients. During these trainings, no patients are at risk if errors are made, and the providers can develop their skills and practice until mastery. However, it is crucial that the providers' performance is measured during training and that feedback is given so that the providers can improve their technique.

### SUMMARY

A system for measuring appropriate ventilatory parameters during training that includes one or more sensors, a memory unit that stores the sensor data, a processing unit that computes the data collected to provide real-time feedback on performance. The sensors may measure temperature, volume of airflow, ventilatory rate, ventilatory pressure, and other relevant properties. The stored data includes historical measurements collected over a period of time by the sensors. The processing unit computes the stored sensor data into performance metrics, compares the metrics against objective measures, and determines what real-time feedback to provide to the user so that the user can improve their skills. This feedback may be provided to the user through a multitude of auditory and visual methods. The system may include a range of standard input and output fittings so that it can be used with any common ventilatory or other airway devices during practice, such as a manual bag valve mask, ventilator, anesthesia gas machine, endotracheal tube, oral airway, or other devices. The system can be used standalone or in combination with another training system. FIG. 1 shows an exemplary system according to some embodiments.

### BRIEF DESCRIPTION OF DRAWINGS

FIG. 1 shows an exemplary system for measuring appropriate ventilatory parameters during training, according to some embodiments;

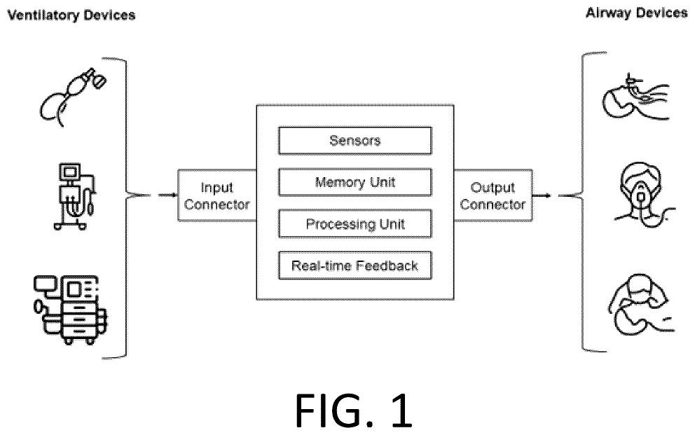

FIG. 2 shows a measurement system 100, according to some embodiments; and

FIG. 3 shows a system 200 that may be utilized to evaluate a medical trainee's ability to perform a ventilatory procedure, according to some embodiments.

### DETAILED DESCRIPTION

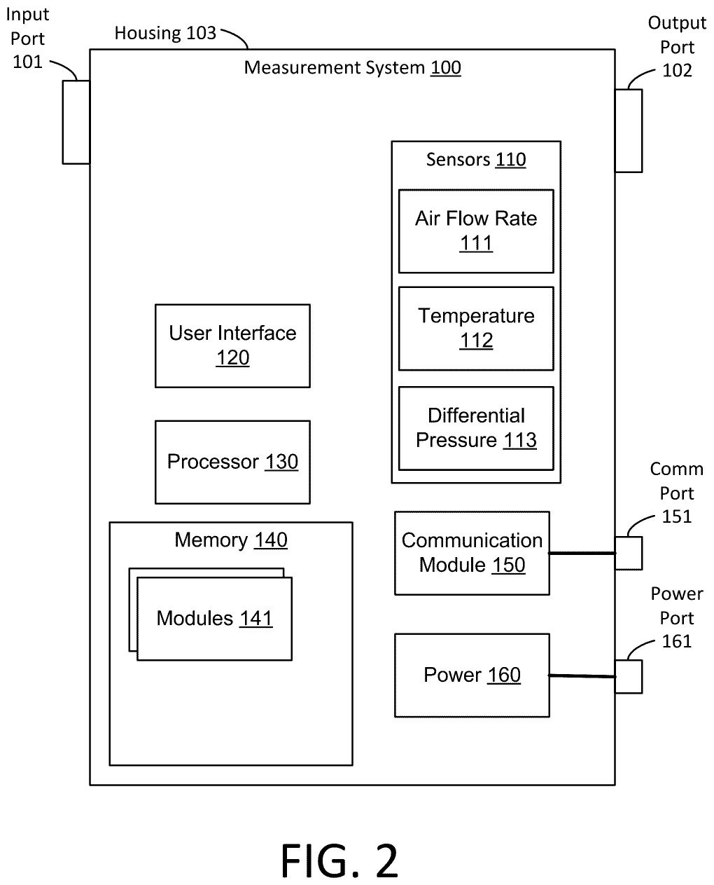

FIG. 2 shows a measurement system 100 according to some embodiments.

In some embodiments, measurement system 100 may be enclosed by a housing 103. Housing 103 may provide mechanical support and protection for the components of measurement system 100.

Measurement system 100 includes an input port 101 for connecting to a ventilatory device. Also, a trainee may act as the “ventilatory device” connected to input port 101 for rescue breathing. The output port 102 may connect to an airway device. The input and output ports may utilize a common (standard) fitting size and adapters may be used if necessary. Examples of ventilatory devices and airways devices include but are not limited to manual bag valve mask, ventilator, anesthesia gas machine, endotracheal tube, and oral airway.

Measurement system 100 may include one or more sensors 110 for measuring properties of the air entering input port 101 and exiting output port 102. These properties may include but are not limited to air flow rate, temperature, and differential pressure, which may be measured by air flow rate sensor 111, temperature sensor 112, and differential pressure sensor 113, respectively. Any suitable sensing technology may be utilized to measure the properties, and the disclosure is not limited to any particular sensor technology.

Processor 130 may be any suitable processing device such as, for example and not limitation, a central processing unit (CPU), digital signal processor (DSP), controller, addressable controller, general or special purpose microprocessor, microcontroller, addressable microprocessor, programmable processor, programmable controller, dedicated processor, dedicated controller, or any suitable processing device. In some embodiments, processor 130 comprises one or more processors, for example, processor 130 may have multiple cores and/or be comprised of multiple microchips. As discussed further below, processor 130 may be configured to implement various algorithms implemented in software, hardware, or a combination thereof and may be operably connected to other components of measurement system 100. Processor 130 may perform such algorithms sequentially, in parallel, or by some other method or combination of methods.

Memory 140 is a non-transitory computer-readable storage media that may be integrated into processor 130 and/or may include “off-chip” memory that may be accessible to processor 130, for example, via a memory bus (not shown). Memory 140 may store software modules 141 that when executed by processor 130 perform desired functions. These functions may interact with the other components of measurement system 100 and/or remote systems. Memory 140 may be any suitable type of non-transitory computer-readable storage medium such as, for example and not limitation, RAM, a nanotechnology-based memory, optical disks, volatile and non-volatile memory devices, magnetic tapes, flash memories, hard disk drive, circuit configurations in Field Programmable Gate Arrays (FPGA), or other semiconductor devices, or other tangible, non-transitory computer storage medium.

In some embodiments a communications module 150 establishes communication with an outside device. Communication may be wired through a communication port 151 or wireless. Communications module 150 may be any suitable software, hardware, or a combination thereof configured to communicate with other devices. In some embodiments, communications module 150 is adapted for direct communication (e.g. USB) or for communication over a network (e.g., TCP/IP). Communications module 150 may be configured to receive instructions from other components measurement system 100 (e.g., from processor 130) to perform communication operations. In some embodiments, communications module 150 may support a custom communication protocol or one or more communications standards or technologies (e.g., Ethernet, Wi-Fi, ZigBee, USB, TCP/IP). In some embodiments communications module 150 is capable of connecting to a remove server via the internet.

Modules 141 may implement algorithms that perform a specific function for measurement system 100. While shown in memory 140, it should be appreciated that the modules may generally be implemented in software, hardware, or a combination thereof. Software modules may comprise computer executable code that when executed by a processor performs certain defined acts.

In some embodiments, modules 141 include a module for collecting measurements from sensors 110. Sensors 110 may include dedicated hardware for digitizing sensor measurements or such digitization may occur as part of the functioning of processor 130. For example, processor 130 may have one or more analog-to-digital converters to digitize sensor measurements. Though this is exemplary, and sensor measurements may be acquired for processing in any suitable way.

In some embodiments modules 141 include a module for calculating performance metrics based on the sensor measurements. These performance metrics may be compared to reference values to determine how effectively a trainee is performing a procedure. Performance metrics and reference values are discussed further herein. In some embodiments sensor measurements are sent for further processing by a remote server via communications module 150.

Measurement system 100 may further comprise a user interface (UI) 120. User interface 120 may include any suitable combination of input and output devices for interfacing with a user. Examples include buttons/keys, touch screens, microphones, displays, lights, speakers and the like. Trainees and trainers may interact with measurement system 100 through UI 120. Though, they may also interact in other ways, for example, through a server connected to communications module 150.

Measurement system 100 may further comprise a power device 160. Power device 160 may include a battery, a power supply, and/or an interface for receiving electrical power. A power port 161 may be utilized to provide power to the device. In some embodiments, power port 161 and communications port 151 may be integrated into a single electromechanical connector (e.g., a USB connector). In some embodiments power port 161 includes a wireless charger. Power device 160 may provide power to the various components of measurement system 100 in accordance with the needs of the various components.

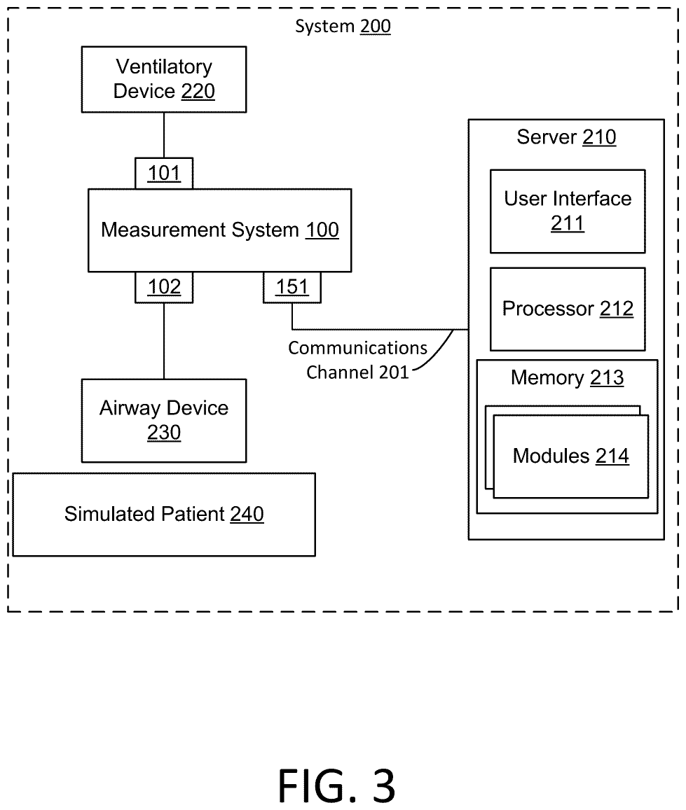

Attention is now turned to FIG. 3 which shows a system 200. System 200 includes a measurement system 100 connected to a ventilatory device 220 and to an airway device 230. Airway device may be placed appropriately on a simulated patient 240. Simulated patient 240 may be, for example a physical, medical mannequin or a digital patient model used for training.

System 200 may be utilized to evaluate a medical trainee's ability to perform a ventilatory procedure using ventilatory device 220.

In some embodiments measurement system 100 is connected to a server 210 via communications channel 201. Communications channel 201 may be a wired or wireless channel. In some embodiments, communications channel 201 connects measurement system 100 to a network (e.g., a LAN, the internet) to which server 210 is connected. In some other embodiments, communications channel 201 connects directly to server 210. Communications channel 201 may be utilized to facilitate real-time communications between measurement system 100 and server 210 during the procedure or may be used to transmit previously recorded data to server 210.

Server 210 may include UI 211, processor 212 and memory 213. UI 211, processor 212 and memory 213 may be similar to the UI, processor and memory described in connection with FIG. 2. However, the components of a specific embodiment need not be the same. As one illustrative example, measurement system 100 may utilize an 8-bit microcontroller while server 210 may utilize a state-of-the-art microprocessor.

Modules 214 may implement algorithms that perform a specific function for measurement system 100. While shown in memory 213, it should be appreciated that the modules may generally be implemented in in software, hardware, or a combination thereof. Software modules may comprise computer executable code that when executed by a processor performs certain defined acts.

Prior to performing a procedure, system 200 may be set up appropriately. Setup may include connecting the appropriate ventilatory device 220 to measurement system 100 (using an appropriate adapter if necessary). Setup may include connecting an appropriate airway device to measurement system 100. Setup may include configuring the measurement system 100 to collect the desired measurements for calculating performance parameters. Measurement setup may include specifying any sensor parameters appropriate for the sensor and procedure. This could include for example and not limitation specifying data rates, ADC resolutions, any filtering or time averaging that may be applied, and any calibration steps that may need to be performed. Setup could include providing a suitable power source (e.g. plugging into the wall if needed). Setup could include establishing a connection to server 210.

Setup could also include specifying performance parameters that are to be calculated based on the sensor measurements and the reference thresholds against which the performance parameters are to be compared. Setup could also include any response that will be presented through simulated patient 240 (e.g., chest rising and falling). Setup could also include determining what outputs of the UI 120 and/or UI 211 will be utilized during the procedure and what information they will provide. For example, the system could provide real-time feedback to the trainee (and a trainer) during the procedure, the system could provide real-time feedback to a trainer and not the trainee during the procedure, or the system may not provide any real-time feedback and all data is only available to the trainer and/or trainee after the procedure has been completed. These settings could be established through pre-specified selections stored in memory or may be adjustable, for example, using UI 120 or UI 211.

During the procedure measurement data is collected from the sensors 110 in measurement system 100 in accordance with the system setup. The measurement data may be immediately communicated to server 210 and/or stored locally by measurement system 100. In some embodiments the measurement data is processed, presented in real-time through the appropriate UI and promptly discarded. In some other embodiments the sensor data is saved for later retrieval. In some embodiments the data is utilized to affect other properties of simulated patient 240 that may simultaneously be measured and/or presented to the trainee. For example, a heart rate of the simulated patient, blood oxygen level (“SpO2”) of the simulated patient, and/or other vitals/measures may be presented to the trainee to provide realistic feedback depending upon the clinical scenario being simulated.

The measurement data may be utilized to calculate performance parameters within measurement system 100 or within server 210. The performance parameters may be compared to the reference thresholds to generate appropriate output that is provided through the UI (e.g., either or both UI 120 and UI 211). For example, a performance parameter may have an upper threshold, a lower threshold or both. The UI may provide a visual or audible cue to indicate that a threshold has been exceeded.

In some embodiments data is saved in association with a particular trainee, and the trainee may re-perform the procedures over time. This information may be utilized to identify trainees that require additional training or to recommend how often re-training for trainees in general or a particular trainee should take place. For example, after performing a series of re-training exercises the system may recommend a time-frame for a next re-training with trainees showing relatively greater retention of performing the procedure within thresholds requiring less frequent training and trainees showing relatively less retention requiring more frequent training.

Representative performance metrics include but are not limited to flow rate itself (e.g., determined directly from a flow rate measurement), the number of “breaths” per unit time (e.g., breaths per minute) administered (e.g., determine by counting the rate of peaks in the air flow rate), the total volume of air administered during each breath (e.g., air flow rate integral), and the quality of the seal of airway device (e.g., implied by the differential pressure measurement). As suggested above, the specific performance metrics and the associated thresholds may be specific to the particular procedure or clinical requirements to which the trainee is being trained. In some embodiments, performance metrics involve further processing of the sensor data such as time averaging or other filtering to prevent false calls or unnecessary alarms.

To further the example of the “quality of the seal of airway device” performance metric, the system may compare the differential pressure measured by differential pressure sensor 113 to a reference threshold. If the differential pressure is below the reference threshold the system outputs an indication that the seal is unacceptable via user interface 120 or 211. If the differential pressure is at or above the reference threshold the system outputs an indication that the seal is acceptable via user interface 120 or 211. The reference threshold may be, for example and not limitation, a value above 0 cmH2O or a value equal to, above or below 1, 2, 3, 4, 5, 6, 7, 8, 9, or 10 cmH2O. However, these are just illustrative examples. The system may be configurable so that any suitable threshold may be set. In some embodiments the systems determines the mask is “on” once a differential pressure is measurable (e.g., above 0 cmH2O), determines the seal is adequate for differential pressure measurements above 5 and below 20 cmH20, and determines that differential pressure measurements at or above 20 cmH2O indicates a potentially dangerous situation. This determination may be output through the user interface.

In further illustrate the example of the “total volume of air administered during each breath” performance metric, representative threshold values may be 240 mL for neonate/infant, 500 mL for pediatric, and 1600 mL for adult. Thresholds may be set for example at, above, or below these representative thresholds by an appropriate value. The appropriate value may be, for example, 0, 1, 2, 3, 4, 6, 7, 8, 9, 10, . . . 28, 28, or 30% or more above or below the representative threshold. Though these are simply illustrative examples of how this threshold may be set. The system may be configurable so that any suitable threshold may be set.

To further illustrate the example of the “number of breaths per unit time”

performance metric, normal ranges may be 30-60 breaths per minute (BPM) for neonate/infant, 15-30 BPM for pediatric, and 10-15 BPM for adult. High and low reference thresholds may be used to indicate the ventilatory rates are above or below these ranges. One or more thresholds may be set for example at, above, or below 10, 11, 12, . . . 68, 69, or 70 BPM. Though these are simply illustrative examples of how this threshold may be set. The system may be configurable so that any suitable threshold may be set.

In some embodiments, temperature measurements are used as a method for determining air flow. Temperature measurements may also be utilized in determining accurate air flow and volume measurements based upon environmental factors (e.g. PV=nrT).

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Accordingly, the foregoing description and drawings are by way of example only.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers, LEDs, colorimetric displays, display screens for visual presentation of output and speakers or other sound generating devices for audible presentation of output. Examples of input devices that can be used for a user interface include keyboards, buttons, switches, or capacitive touch interfaces, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

In this respect, it should be appreciated that one implementation of the above-described embodiments comprises at least one computer-readable medium encoded with a computer program (e.g., a plurality of instructions), which, when executed on a processor, performs some or all of the above-discussed functions of these embodiments. As used herein, the term “computer-readable medium” encompasses only a computer-readable medium that can be considered to be a machine or a manufacture (i.e., article of manufacture). A computer-readable medium may be, for example, a tangible medium on which computer-readable information may be encoded or stored, a storage medium on which computer-readable information may be encoded or stored, and/or a non-transitory medium on which computer-readable information may be encoded or stored. Other non-exhaustive examples of computer-readable media include a computer memory (e.g., a ROM, a RAM, a flash memory, or other type of computer memory), a magnetic disc or tape, an optical disc, and/or other types of computer-readable media that can be considered to be a machine or a manufacture.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

For the purposes of describing and defining the present disclosure, it is noted that terms of degree (e.g., “substantially,” “slightly,” “about,” “comparable,” etc.) may be utilized herein to represent the inherent degree of uncertainty that may be attributed to any quantitative comparison, value, measurement, or other representation. Such terms of degree may also be utilized herein to represent the degree by which a quantitative representation may vary from a stated reference (e.g., about 10% or less) without resulting in a change in the basic function of the subject matter at issue. Unless otherwise stated herein, any numerical values appeared in this specification are deemed modified by a term of degree thereby reflecting their intrinsic uncertainty.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A system comprising:
an input port for connecting to a ventilatory device;
an output port for connecting to an airway device;
a plurality of sensors for measuring properties of the air passing in between the input port and output port;
a user interface output device; and
at least one processor and at least one non-transitory computer-readable storage medium having stored thereon instructions which, when executed, program the at least one processor to perform a method comprising:
receiving measurements from the plurality of sensors;
generating performance metrics based on the measurements;
comparing the performance metrics to one or more reference thresholds; and
outputting via the user interface output device an indication of performance based on the comparing.

2. The system of claim 1, wherein the plurality of sensors include a temperature sensor.

3. The system of claim 1, wherein the plurality of sensors include an air flow rate sensor.

4. The system of claim 1, wherein the plurality of sensors include a differential pressure sensor.

5. The system of claim 1, wherein the user interface output device is a speaker and a sound is output therefrom indicating the performance.

6. The system of claim 1, wherein the user interface output device is a light and a color of the light output therefrom indicates the performance.

7. The system of claim 6, where in a green light indicates performance is within the reference threshold and a red light indicates performance is beyond the reference threshold.

8. The system of claim 1, further comprising a housing having the input port and output port as ports thereto and the plurality of sensors are within the housing.

9. The system of claim 8, wherein the housing has dimensions that do not exceed 6 inches in width, length and height.

10. The system of claim 9, wherein the input port, output port, and plurality of sensors are within a housing having dimensions that does not exceed 4 inches in width, length and height.

11. The system of claim 8, further comprising a communications module within the housing, the at least one processor and the at least one non-transitory computer-readable storage medium are outside the housing, and the communications module communicates the sensor measurements to the at least one processor.

12. The system of claim 11, wherein the at least one processor, the at least one non-transitory computer-readable storage, and the user interface output device are part of a computer system that is not mechanically attached to the housing.

13. The system of claim 8, wherein the at least one processor, the at least one non-transitory computer-readable storage, and the user interface output device are mechanically secured to the housing.

14. The system of claim 1, wherein the plurality of sensors include a differential pressure sensor and the generating performance metrics based on the measurements includes determining a seal between the airway device and a simulated patient is acceptable if a measurement of differential pressure is above a threshold.
